(19) United States
(12) Patent Application Publication
(10) Pub. No.: US 2026/0345398 A1
(43) Pub. Date: Nov. 5, 2026

(54) A COAGULATION-BASED SANITARY PRODUCT

(71) Applicant: Shagun Pankaj MAHESHWARI, Mumbai, Maharashtra (IN)

(72) Inventor: Shagun Pankaj MAHESHWARI, Mumbai, Maharashtra (IN)

(21) Appl. No.: 19/488,008

(22) PCT Filed: May 25, 2024
(86) PCT No.: PCT/IB2024/055110

§ 371 Date: Nov. 25, 2025

(30) Foreign Application Priority Data
May 26, 2023 (IN) 202321036615

(51) Int. Cl. A61F 13/537; A61F 13/53

(52) U.S. Cl. CPC A61F 13/537; A61F 2013/53062; A61F 2013/530802

(57) ABSTRACT

The present invention discloses a biodegradable and compostable sanitary product (300) for wearing against a body of a user. The sanitary product (300) comprising at least one permeable coagulating core layer (300), wherein the at least one permeable coagulating core layer (300) comprises an intrinsic bulk volume, and containing a blood coagulating agent (301) present within said bulk volume, said blood coagulating agent is configured to enable coagulation of a menstrual discharge.

### CROSS-REFERENCE TO RELATED APPLICATIONS

The present application is a National Stage Application of PCT International Application No.: PCT/IB 2024/055110 filed on May 25, 2024, which claims priority to Indian Patent Application 202321036615, filed with the Indian Patent Office on May 26, 2023, the disclosures of which are incorporated herein by reference in their entirety.

### FIELD OF INVENTION

The embodiments of the present disclosure generally relate to articles designed to collect and contain blood or menstrual discharge, such as sanitary napkins or pads.

### BACKGROUND

Background description includes information that may be useful in understanding the present invention. It is not an admission that any of the information provided herein is prior art or relevant to the presently claimed invention, or that any publication specifically or implicitly referenced is prior art.

The primary objective of menstrual pads is to collect and contain menstrual discharge. A secondary objective is to keep the skin that's in contact with the pad dry from discharge.

Common art menstrual pads achieve these goals by absorbing the menstrual discharge into an absorbing material. The best absorbing material (“Super Absorbant Polymers”, or SAPs) are plastic based, and this presents two problems: From a health perspective, plastics leach chemicals such as Phtalates, which are subsequently absorbed by the skin, and from an environmental perspective, plastics degrade into persistent microplastics which have a negative impact on the local watersheds and food chains. There are existing natural-fiber plastic-free alternatives to SAPs, but they do not absorb as well and result in inferior product functionality.

Menstrual pads generally comprise several layers, parallel to the skin, as depicted in FIGS. 1a and 1b. Proximal to the body is a soft “top sheet” layer [101] which emphasizes wicking across its thickness. Most distal from the body is a leakproof “back sheet” [104] which blocks menstrual fluid and prevents leakage towards any undergarments worn over the pad. In between the top sheet and back sheet is the main containment layer called the “core” [103], and typically a permeable “lateral distribution layer” or “transport layer” [102] which helps spread the discharge laterally into all regions of the core. Typically, the core is distal to the lateral distribution layer. Other layers may also be present. The general direction of flow of menstrual discharge is depicted by arrows [105]. And [106] is the wearers undergarment.

Menstrual pads are generally shaped in order to conform to the body, and to be compatible with various garments that may be worn over them. Sometimes they include “wings” which help them adhere to those garments. Several such shapes are depicted in FIG. 2.

FIG. 2 depicts examples of potential shapes and designs of the proposed biodegradable sanitary pad. Certain embodiments of the invention the proposed biodegradable sanitary pad may be of sizes that conform to the wearer's undergarment or requirement such as volume of menstrual fluid to be held. FIG. 2A shows a regular long product form factor, FIG. 2B shows a form factor with “wing” like protrusions that are useful to hold it in place. FIG. 2C shows a smaller design that is meant for lighter flow days. FIGS. 2D-2E show slightly wider bottoms for all night and extended use comfort for the wearer as these have larger surface areas.

In an embodiment, of the invention have side panels that fold outwards to form a “wing” like structure (as shown in FIGS. 2B and 2E) that the user can use to secure it to their undergarment. The wings may also have a glue coating that helps with positioning and gives the user a safe and secure feeling.

Further, tampons and cups are cylindrical in shape and designed to be insertable. Tampons absorb the blood into their bulk, whereas menstrual cups merely contain it. Generally, Tampons can be thought of as “rolled up pads”, comprised of concentric rather than laminar layers, where the outermost layer of a tampon corresponds to the top sheet of a pad.

Menstrual pads generally comprise several laminar or flat layers, parallel to the skin. Proximal to the body is a “top sheet” layer which is configured to emphasize wicking across its thickness. Most distal from the body is a “back sheet” which is configured to block menstrual discharge and prevents leakage towards any undergarments worn over the pad. In between the top sheet and the back sheet is the main containment layer which is absorbent and is called the “core”, and typically an additional permeable “lateral distribution layer” or “transport” layer which helps spread the discharge laterally into various regions of the core. Typically, the core is distal to the transport layer. Other layers may also be present.

There is therefore a need in the art to provide healthier and more eco-friendly sanitary napkins that can overcome the shortcomings of the existing prior art.

### OBJECTIVES OF THE PRESENT DISCLOSURE

Some of the objectives of the present disclosure, which at least one embodiment herein satisfies, are as listed herein below:

An objective of the present disclosure is to provide a healthier plastic-free sanitary product able to collect and contain menstrual discharge as well as a SAP-based one.

Another objective of the present disclosure is to provide a biodegradable plastic-free sanitary product able to collect and contain menstrual discharge as well as a SAP-based one.

Another objective of the present disclosure is to provide a comfortable plastic-free sanitary product able to collect and contain menstrual discharge as well as a SAP-based one.

### SUMMARY

The present invention relates the present disclosure generally relate to absorbent articles, such as sanitary napkins. More particularly, the present disclosure relates to a biodegradable and compostable sanitary product with a haemostatic core.

An aspect of the present disclosure provides a coagulation based sanitary product for wearing against a body. The sanitary product comprising at least one coagulating core layer. The at least one coagulating core layer comprises an intrinsic bulk volume, and containing a blood coagulating agent present within said bulk volume, said blood coagulating agent configured to enable coagulation of a menstrual discharge.

In an aspect, the at least one coagulating core layer comprises a fabric comprising one or more fibers having intrinsic bulk volumes, and said blood coagulating agent present within the bulk volume of the one or more fibers.

In an aspect, the said fabric is a non-woven fabric.

In an aspect, the sanitary product further comprising a lateral transport layer adjacent to at least one coagulating core layer and at an overlapping position, said lateral transport layer is positioned distal to the body of the user from at least one coagulating core layer.

In an aspect, the at least one coagulating core layer further comprises one or more regions extending through a thickness of said at least one coagulating core layer, and configured to allow the flow of the menstrual discharge through said one or more regions and into said lateral transport layer.

In an aspect, the one or more regions are holes.

In an aspect, the said one or more regions are holes filled with a wicking material.

In an aspect, the coagulating agent comprises at least one material selected from any or a combination of chitosan, chitosan salts, chitosan lactate, chitosan acetate, calcium alginate, calcium ions, other alginates.

In an aspect, the at least one coagulating core layer further contains a plasticiser configured to enhance compliance of the at least one coagulating core layer.

In an aspect, said plasticiser is selected from any or a combination of glycerol, other non-endocrine disrupting, non-phthalate plasticisers.

In an aspect, said fibers are selected from any or a combination of cellulose, cotton, hemp, natural fibers.

Various objects, features, aspects, and advantages of the inventive subject matter will become more apparent from the following detailed description of preferred embodiments, along with the accompanying drawing figures in which like numerals represent like components.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIGS. 1A and 1B illustrates different layers of an existing sanitary product.

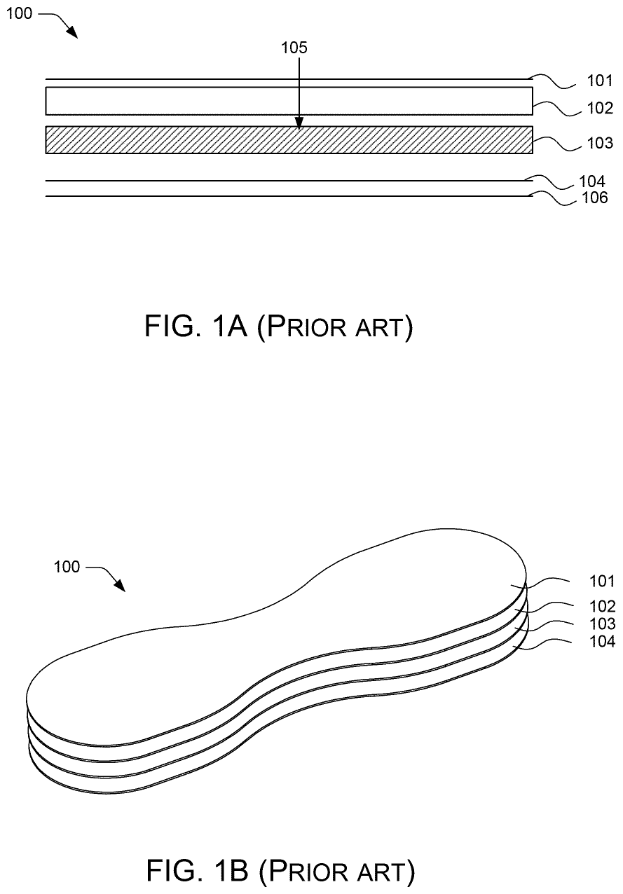

FIGS. 2A-2E illustrate exemplary representations of different shapes and designs of the existing sanitary product.

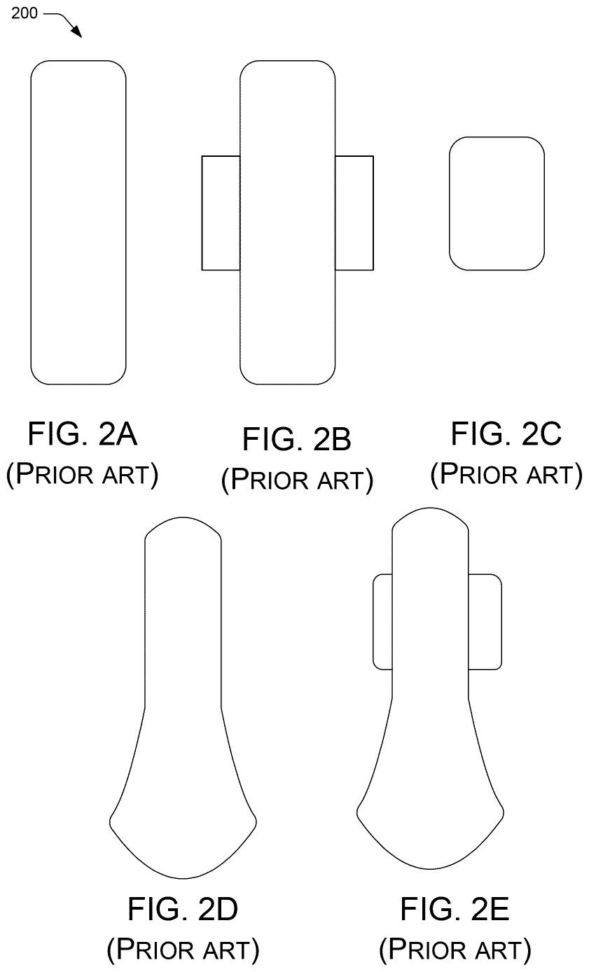

FIG. 3 illustrates an exemplary representation of a proposed coagulating core based sanitary pad, in accordance with an embodiment of the present disclosure.

FIGS. 4A-4B illustrate exemplary representation of combination of the core and transport layers of the proposed coagulating core sanitary pad, in accordance with embodiments of the present disclosure.

FIGS. 5A-5B illustrate exemplary representations of a tampon constructed as a rolled up version of pad of the proposed coagulating core sanitary pad, in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

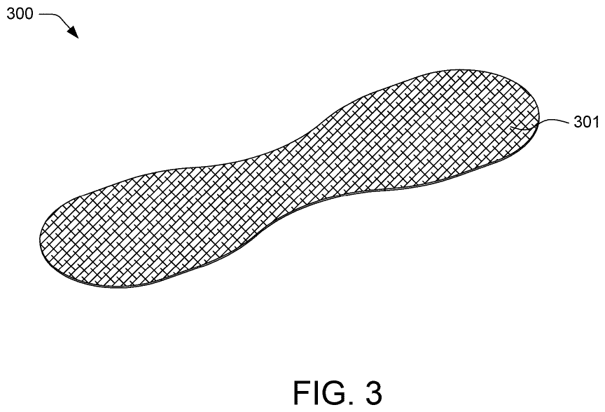

Various aspects of the present disclosure are described with respect to FIGS. 3 to 5A-5B.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Various terms are used herein. To the extent a term used in a claim is not defined, it should be given the broadest definition persons in the pertinent art have given that term as reflected in printed publications and issued patents at the time of filing.

The primary objective of menstrual pads is to collect and contain menstrual discharge. The pad is used by placing it so it covers the vulva, directly against the skin. A secondary objective is to keep the skin that's in contact with the pad dry from discharge.

The present disclosure provides sanitary pad which is generally made from natural materials, is plastics-free, and can collect and contain menstrual discharge as proficiently as plastics-based pads while providing a dryer feeling against the skin.

FIG. 3 illustrates an exemplary representation of a proposed coagulating core based sanitary pad, in accordance with an embodiment of the present disclosure.

In an embodiment, the core layer FIG. 3 is permeable 300 and contains a coagulating agent 301 or coagulant inside its bulk volume so the pad collects and contains the menstrual discharge by means of coagulating it inside the bulk volume of the core containment layer. This is in contrast with existing art pads that contain the menstrual discharge by means of absorbance only. (Other layers which may be present in the complete pad are not shown in FIG. 3.)

In an embodiment, blood naturally has the inherent ability to coagulate. A coagulating agent or coagulant is a chemical that triggers the coagulation response of the blood. There are many coagulants in prior art, and they are commonly used in products such as coagulating or clotting bandages.

However, the purpose of clotting bandages is to stem the flow of blood, and cause coagulation inside the wound or on the wearer's skin. In contrast, a menstrual pad must not interfere with the outflow of menstrual discharge—it first needs to wick the menstrual discharge away from the skin, and then coagulate it in the core layer, while allowing additional menstrual discharge to outflow from the body for many hours. For this reason, clotting bandages emphasize locating a layer of coagulant proximal or adjacent to the skin or wound, whereas a pad must provide a specific structure to avoid this, and the structure of clotting bandage is inadequate for a menstrual pad. Unlike a bandage, a menstrual pad needs to remain soft and compliant and allow for daily activities by the wearer without discomfort. For this reason, the menstrual pad needs to enmesh with the clotted blood, preventing the formation of a contiguous and rigid blood clot.

In an embodiment, the core layer comprises a permeable substance containing a coagulating agent within its bulk volume.

“Bulk volume” is all the space occupied by the material, including any micro-cavities within itself, but excluding its external facing surface. For a sponge layer, for example, the bulk volume includes the base material and the internal voids, but excludes the outer surface of the sponge. For a layer made of non-woven fabric made of many short fibers, the bulk volume includes the fibers and the spaces between them, but not the outer surface of the fabric as a whole. For a twisted round yarn, for example, which is typically comprised of many micro-fibers wound together, the bulk volume is the cylindrical volume comprising the micro fibers and the spaces between them.

In an embodiment, a coagulant that merely coats the outer surface of a layer is not considered to occupy the bulk volume of the layer. If blood coagulates on the outer surface of a material, it is able to form a contiguous solid and thus becomes hard and brittle. If blood coagulates inside the bulk volume of a permeable material it is interrupted by the inner structure of the material and so remains softer and more compliant.

In an embodiment, introducing a coagulant into the bulk volume of a material can be done for example by dissolving the coagulant into a solution, soaking the material in the solution, and then squeezing it out or letting it dry. If the material is comprised of smaller components, such as fabrics comprised of fibers, it is possible to introduce the coagulant into the components before aggregating them into the bulk material.

In another embodiment, the base material of the coagulating core is a permeable or spongy material (such as a Silicone-based, cellulose or natural sea sponge, or sintered material), and the coagulant is infused, soaked, or otherwise introduced into the bulk volume of the core.

In another embodiment, the base material of the coagulating core is a woven fabric made of plastic-free yarns (such as cellulose, cotton, hemp, viscose, flax, kenaf, jute or other natural fibers) which are cellulose based plant fibers,). The coagulating agent is then infused, soaked, or otherwise introduced into the bulk volume of the core or the bulk volume of the yarn.

In another embodiment, the base material of the coagulating core is a non-woven fabric made of plastic-free fibers or yarns (such as cellulose, cotton, or hemp, viscose, flax, kenaf, jute cupor other natural fibers). The coagulating agent is infused, soaked, or otherwise introduced into the bulk volume of the constituent fibers or yarns before they are aggregated into the fabric.

In embodiments of this invention, the coagulant comprises at least one of Chitosan (from chitin) compounds, such as Chitosan Lactate and Chitosan Acetate. Other natural and sustainable blood coagulants may also be used in place of Chitosan, such as pectin, calcium ions, diatomaceous earth, and extracts from moringa seeds, banana peel, jatropha curcas, cassava peel starch, watermelon, pawpaw, beans, and Nirmali seeds.

As a polysaccharide, Chitosan can also naturally form a rigid natural and biodegradable structural material that is sometimes used in other applications as a biopolymer, but such forms are not compatible with it being introduced inside the bulk volume of a permeable substance, and so are not useful in embodiments of this invention.

In embodiments of this invention, in order to enhance the softness and compliance of the base material of the core layer, the layer further contains a plasticiser such as (but not limited to) glycerol.

In another embodiment of this invention, increased softness and compliance of the coagulating core layer's fabric is achieved by shredded it after the coagulant is introduced into its bulk volume, and then re-aggregating the shredded pieces.

In an embodiment, a core layer that works by coagulation has the property that when it saturates, it is no longer permeable to menstrual discharge, since it is congealed in place, clogged, and dry.

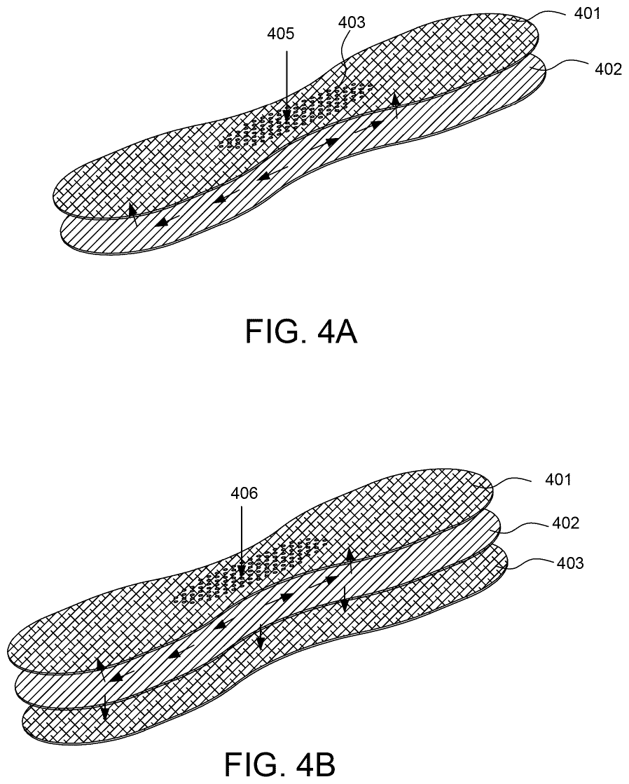

FIGS. 4A-4B illustrate exemplary representation of combination of the core and transport layers of the proposed coagulating core sanitary pad, in accordance with embodiments of the present disclosure.

In another embodiment, as shown in FIG. 4A, the core layer 401 is located proximal to the body relative to the transport layer 402 (opposite of existing art pads), and has an array of holes or wicking regions 403 in it allowing it to transport menstrual discharge across its thickness even when the rest of its bulk is saturated and clogged. The wicking regions can be implemented for example as holes that contain a non-coagulating permeable material. (Other layers which may be present in the complete pad are not shown in FIGS. 4A and 4B).

The advantage of this configuration is that most of the layer closer to the body remains dry, while the transport layer which is inherently wet continues to function, dispersing the menstrual fluid along the distal surface of the core layer. Blood flow is depicted as arrows 405.

In another embodiment, as shown in FIG. 4B, a second coagulating core layer 404 is further added on the distal side of the transport layer, this second core layer configured without any holes. Blood flow is depicted as arrows 406.

In another embodiment, this configuration ensures that the transport layer is sandwiched between two coagulating core layers and the pad presents dryer surface both in the direction of the skin and in the direction of any garments worn over the pad.

These two embodiments cannot function with an absorbance-based core layer since it remains inherently wet.

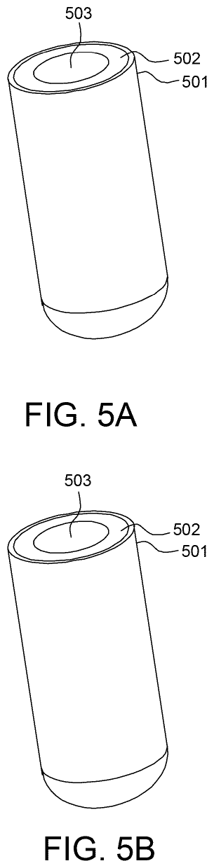

FIGS. 5A-5B illustrate exemplary representations of a tampon constructed as a rolled up version of pad of the proposed coagulating core sanitary pad, in accordance with embodiments of the present disclosure.

In another embodiment, the top sheet layer of the pad corresponds to the outmost layer of the tampon 501. The leakproof layer of the pad does not exist in the tampon.

The lateral transport layer of the pad resides inside the tampon, and is responsible for transport of menstrual discharge in the axial direction of the cylinder 502. As in the pad, the coagulating core layer can be external or internal to the transport layer 503.

In the embodiment depicted in FIG. 5A, the coagulating core layer is at the center, corresponding to a standard layer structure, so the transport layer surrounds the core layer.

In the embodiment depicted in FIG. 5B, the transport layer is at the center. Since the blood is arriving axially, from above the tampon, this configuration does not require the array of holes that the laminar coagulation layer of the pad needed.

In an embodiment, the present disclosure provides sanitary pad which is generally made from natural materials, is plastics-free, and can collect and contain menstrual discharge as proficiently as plastics-based pads while providing a dryer feeling against the skin.

In an embodiment, the pad collects and contains the menstrual discharge by means of coagulating it inside the bulk of a containment layer (“Core”) using a coagulating agent. This is in contrast with existing art pads that contain the menstrual discharge by means of absorbance only.

In an embodiment, blood already has the inherent ability to coagulate. A coagulating agent is a material that triggers the coagulation response of the blood. There are many coagulation agents in prior art, and they are commonly used in products such as coagulating or clotting bandages.

However, the purpose of clotting bandages is to stem the flow of blood, and cause coagulation inside the wound or on the wearer's skin. In contrast, a menstrual pad must not interfere with the outflow of menstrual discharge—it first needs to wick the menstrual discharge away from the skin, and then coagulate it in the core layer, while allowing additional menstrual discharge to outflow from the body for many hours. Unlike a bandage, a menstrual pad needs to remain soft and compliant and allow for daily activities by the wearer without discomfort. For this reason, clotting bandages emphasize placing a layer of coagulating agent proximal to the skin, whereas a pad must provide a specific structure to avoid this, and the structure of clotting bandage is inadequate for a menstrual pad.

In an embodiment, the core layer comprises a permeable layer.

In an embodiment, the present disclosure provides a sanitary product for wearing against a body. The sanitary product comprising at least one coagulating core layer. The at least one coagulating core layer comprises an intrinsic bulk volume, and containing a blood coagulating agent present within said bulk volume, said blood coagulating agent configured to enable coagulation of a menstrual discharge.

In an embodiment, the at least one coagulating core layer comprises a fabric comprising one or more fibers having intrinsic bulk volumes, and said blood coagulating agent present within the bulk volume of the one or more fibers.

In an embodiment, the said fabric is a non-woven fabric.

In an embodiment, the sanitary product further comprising a lateral transport layer adjacent to at least one coagulating core layer and at an overlapping position, said lateral transport layer is positioned distal to the body of the user from at least one coagulating core layer.

In an embodiment, the at least one coagulating core layer further comprises one or more regions extending through a thickness of said at least one coagulating core layer, and configured to allow the flow of the menstrual discharge through said one or more regions and into said lateral transport layer

In an embodiment, the one or more regions are holes.

In an embodiment, the said one or more regions are holes filled with a wicking material.

In an embodiment, the coagulating agent comprises at least one material selected from any or a combination of chitosan, chitosan salts, chitosan lactate, chitosan acetate, calcium alginate, calcium ions, other alginates.

In an embodiment, the at least one coagulating core layer further contains a plasticiser configured to enhance compliance of the at least one coagulating core layer.

In an embodiment, said plasticiser is selected from any or a combination of glycerol, other non-endocrine disrupting, non-phthalate plasticisers.

In an embodiment, said fibers are selected from any or a combination of cellulose, cotton, hemp, natural fibers.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” may include plural reference unless the context clearly dictates otherwise. Also, as used in the description herein, the meaning of “in” may include “in” and “on” unless the context clearly dictates otherwise.

It is to be appreciated by a person skilled in the art that while various embodiments of the present disclosure have been elaborated for a biodegradable and compostable sanitary product for wearing against a body of a user. However, the teachings of the present disclosure are also applicable for other types of applications as well, and all such embodiments are well within the scope of the present disclosure. However, the biodegradable and compostable sanitary product is also equally implementable in other industries as well, and all such embodiments are well within the scope of the present disclosure without any limitation.

Accordingly, the present disclosure provides a biodegradable and compostable sanitary product.

Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as limitation.

### Advantages of the Present Disclosure

The present disclosure provides provide a low-cost and comfortable sanitary pad that is biodegradable, and hence sustainable to the environment.

The present disclosure provides a sustainable one-time-use biodegradable and compostable sanitary pad.

The present disclosure provides a sanitary pad includes a haemostatic material which consists of antibacterial properties that help prevent infections. Further, once coagulated, the discharge can no longer ooze out of the layer if squeezed mechanically.

The present disclosure provides a sanitary pad which generates fewer greenhouse gas emissions than conventional pads, which are made from synthetic materialporous.

The present disclosure provides for a sanitary pad which is generally made from natural materials such as organic material, which are less likely to cause skin irritation or allergic reactions.

1. A coagulation-based sanitary product for wearing against a body of a user, the sanitary product comprising:
at least one permeable coagulating core layer, wherein the at least one permeable coagulating core layer comprises an intrinsic bulk volume, and containing a blood coagulating agent present within said bulk volume, said blood coagulating agent is configured to enable coagulation of a menstrual discharge.

2. The sanitary product as claimed in claim 1, wherein the at least one coagulating core layer comprises a porous material having intrinsic bulk volumes, and said blood coagulating agent present within the bulk volume of said porous material

3. The sanitary product as claimed in claim 2, wherein the porous material consists of (Silicone based sponge, cellulose-based sponge, natural sponge, sea sponge).

4. The sanitary product as claimed in claim 1, wherein the at least one coagulating core layer comprises a fabric comprising one or more fibers having intrinsic bulk volumes, and said blood coagulating agent present within the bulk volume of the one or more fibers.

5. The sanitary product as claimed in claim 4, wherein said fabric is a non-woven fabric.

6. The sanitary product as claimed in claim 2, wherein said one or more fibers are selected from any or a combination of cellulose, cotton, hemp, natural fibers.

7. The sanitary product as claimed in claim 1, wherein the coagulating agent comprises at least one material selected from any or a combination of chitosan, chitosan salts, chitosan lactate, chitosan acetate, calcium alginate, calcium ions, other alginates.

8. The sanitary product as claimed in claim 1, wherein the sanitary product further comprising at least one lateral transport layer adjacent to said at least one coagulating core layer and at an overlapping position, said at least one lateral transport layer is positioned distal to the body of the user from said at least one coagulating core layer.

9. The sanitary product as claimed in claim 8, wherein said at least one coagulating core layer further comprises one or more regions extending through the thickness of said at least one coagulating core layer, and configured to allow the flow of the menstrual discharge through said one or more regions and into said at least one lateral transport layer.

10. The sanitary product as claimed in claim 9, wherein said one or more regions are holes.

11. The sanitary product as claimed in claim 9, wherein said one or more regions are holes filled with a wicking material.
